(19) United States
(12) Patent Application Publication
(10) Pub. No.: US 2015/0201870 A1
(43) Pub. Date: Jul. 23, 2015

(54) Gastric Acid Detector

(71) Applicant: Daniel Gelbart, Vancover (CA)

(72) Inventor: Daniel Gelbart, Vancover (CA)

(21) Appl. No.: 14/599,208

(22) Filed: Jan. 16, 2015

Related U.S. Application Data
(60) Provisional application No. 61/928,706, filed on Jan. 17, 2014.

(51) Int. Cl. A61B 5/1473; A61B 5/145; A61B 5/00

(52) U.S. Cl. CPC A61B 5/1473; A61B 5/6852; A61B 5/7405; A61B 5/742; A61B 5/14507

(57) ABSTRACT

In medical procedures requiring access to the stomach, such as insertion of a nasogastric tube (also known as NG tube), verification is required that the tube has indeed reached the stomach and did not find its way into another organ such as the lungs. The invention provides a simple and foolproof way of establishing that the stomach was reached. The method does not require any additional training of the medical staff. The invention can be used in conjunction with an NG tube and provides feedback to the user when the NG tube is correctly inserted in the stomach by detecting the presence of Gastric acid.

### BACKGROUND OF THE INVENTION

In medical procedures requiring access to the stomach, such as insertion of a nasogastric tube (also known as NG tube), verification is required that the tube has indeed reached the stomach and did not find its way into another organ such as the lungs. The invention provides a simple and foolproof way of establishing that the stomach was reached. The proposed method does not require any additional training for the medical staff.

### BRIEF DESCRIPTION OF DRAWINGS

FIG. 1 is a cross-sectional view an NG tube inserted into a patient.

FIG. 2 is a sectional view of the gastric acid detector in an NG tube.

FIG. 3 is a general view of the gastric detector.

FIG. 4 is a diagram showing the preferred embodiment of the Gastric acid detector.

FIG. 5 is a detailed schematic of the preferred embodiment.

FIG. 6 is a diagram showing an alternative method of detecting the presence of blood.

### PRIOR ART

The current practice is radiographic confirmation, which is expensive and time consuming. Previous to the practice of radiographic confirmation, NG tube placement was confirmed using auscultation of air through the tube. While this is inexpensive and fast, it is impractical for obese patients and is therefore uncommonly practiced in many regions.

Prior art has shown methods of determining the location of the NG tube using various pH detectors, such as U.S. Pat. No. 5,105,812 and U.S. patent application Ser. No. 11/799,644. These pH sensor methods are expensive, tedious and often require additional equipment and, because they are multiuse devices, must be sterilized between uses. Others methods, such as U.S. Ser. No. 12/296,754, have proposed pH sensitive dyes. While these devices are disposable, they are difficult to read and require either a means of visualize the dye at the distal end of the NG tube or aspirating fluid to test. U.S. Pat. No. 5,891,054 also propose a method to test aspirated fluid for the presence of bilirubin, pepsin and tryp sin. Other placement methods, U.S. Ser. Nos. 10/945,758 and 11/139,118, have been described, that will detect carbon dioxide if the NG tube is placed in the lungs.

The disclosed invention provides a means of safely inserting an NG tube that can be completed without transferring the patient, does not require exposure to ionizing radiation, can differentiate between Gastric acid and other body fluids, is inexpensive and, most importantly, is quick to insert and confirm placement. To our knowledge, there is no prior art that meets all of these criteria.

### DESCRIPTION OF THE PREFERRED EMBODIMENT OF THE INVENTION

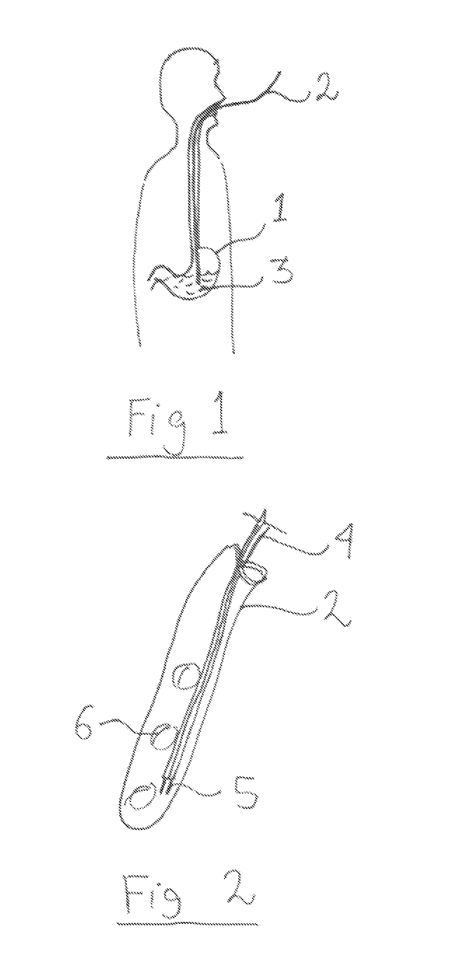

Referring to FIG. 1 and FIG. 2, an NG tube 2 is inserted into stomach 1 of a patient. The detection is based on the fact that the stomach contains Gastric acid 3 (mainly HCl) which is a strong acid and can be detected electrically. Typically, the pH range of a human stomach is between 1 and 5. The preferred embodiment of the invention places a pair of electrical wires 4 into the Gastric tube 2 before inserting the Gastric tube 2 into the body. The ends of wires 4 have a short bare section 5. When the end of tube 2 reaches the stomach, Gastric acid comes into contact with bare wires 5 and creates an electrical path between the wires. Typically, NG tubes have side openings 6. These holes allow the gastric acid to enter tube 2 as soon as it is inserted into the stomach.

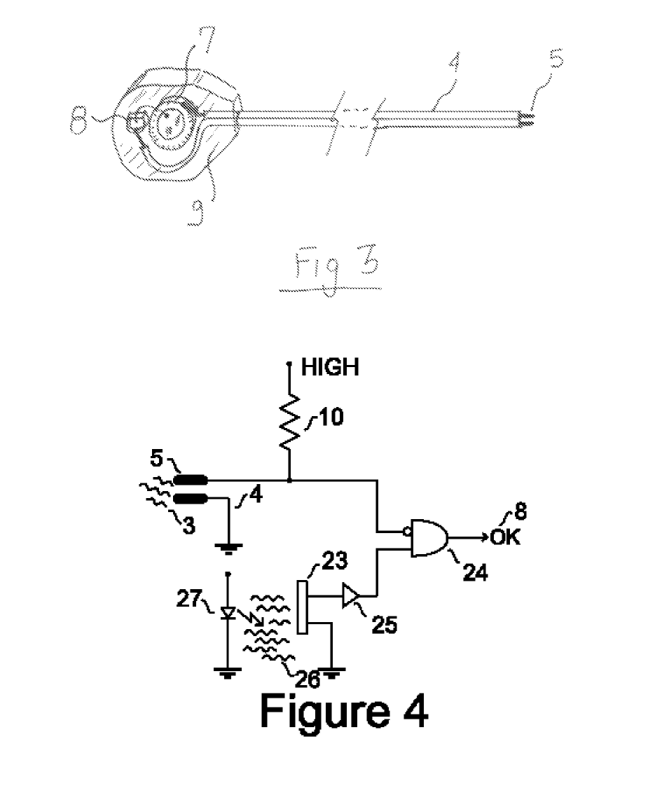

Referring now to FIG. 3 and FIG. 4, the other end of wires 4 is connected to a battery 7 in series with a Light Emitting Diode (LED) 8. LED 8 lights up when the electrical circuit is closed by the gastric acid. LED 8 and battery 7 can be encapsulated or housed in enclosure 9. The LED 8 gives the user permission to feed through the NG tube.

It is desired that the LED will light up only when exposed wire ends 5 contacts gastric acid 3 and not other liquids such as blood, saline, food etc. This can be done by selecting the dimensions of exposed wires 5. Gastric acid typically has a pH between 1 and 5 and is a better conductor than blood, saline or other liquids the gastric tube may encounter. When using a red LED 8 and a 3V lithium button cell (similar to the one used in watches), it was found out that exposed leads 5 should be 2-4 mm long, with a separation of 1-2 mm, in order to have a sufficient current to light up the LED 8 in gastric acid but not in the other liquids. The exact dimensions of exposed leads 5 should be established experimentally. It is also possible to place a resistor parallel to a LED 8 as a way to select the sensitivity. A typical resistor 10 would be in the 1-10 MΩ range.

The continuity conductor method described above will work is many patients, but the pH alone cannot be relied on and can generate a false positive when it is exposed to blood. A more comprehensive detector includes a circuit to differentiate low acidity gastric fluid from blood. Several methods can be used. The preferred embodiment of blood detection would detect the red colour of blood and prevent the “OK” or confirmation LED 8 to go on. One possible method of detecting blood would be to have a blue LED 27 at the distal end of catheter emitting light through the detected liquid 26 to a photodetector 23. If the liquid 26 is red blood, the blue light will be absorbed and the photodetector 23 will be low. Logic gate 24 will therefore be low, even if the exposed leads 5 are conducting, inhibiting the “OK” LED 8 from turning on. If no blood is present, the photodetector 23 will receive light from the LED 27 and the resulting signal will be amplified 25 and allow the “OK” LED 8 to light if, exposed leads 5 are immersed in gastric acid.

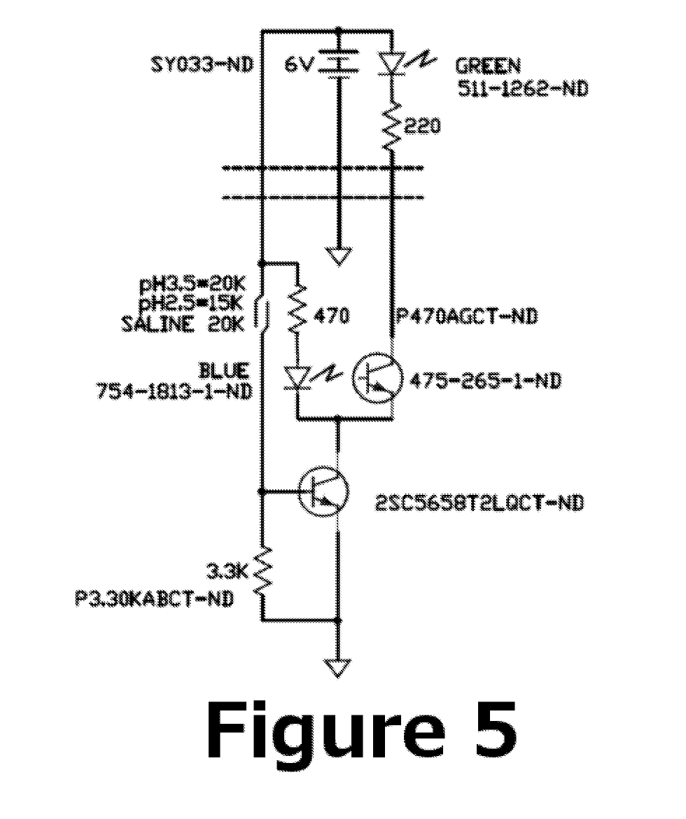

FIG. 5 is a detailed schematic of FIG. 4 which includes the Digikey part numbers.

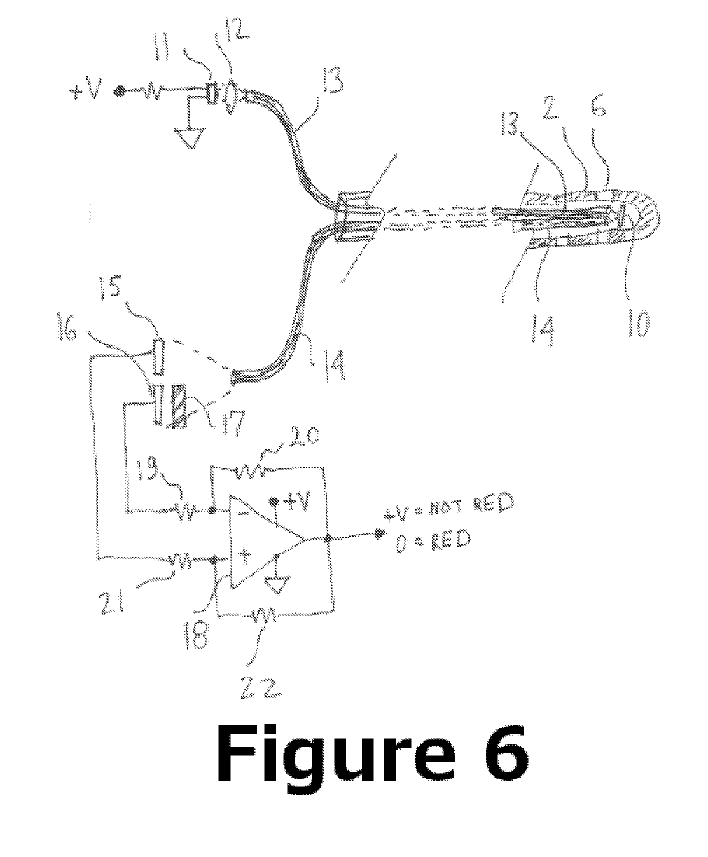

An alternative method of detecting blood in the liquid is presented in FIG. 6: An LED 11 is coupled to a multimode optical fiber 13 by lens 12. Direct coupling, without a lens, is also possible. The fiber 13 can be a low cost plastic type, typically 0.5 mm to 1 mm OD. The light from this fiber shines on a small white spot 10 located at the distal tip of the NG tube 2. The distance between the tip of the fiber and white spot 10 is small, typically less than 1 mm. The reflected light is picked up by an identical optical fiber 14 and used to illuminate detectors 15 and 16. Detector 16 has a red optical band-pass filter 17 in front of it. The exact band pass wavelength is matched to the color that needs to be sensed, about 600 to 650 nm for blood. The outputs of detectors 15 and 16 are fed to amplifier 18. The gain of the inputs is set by resistors 19, 20 and 21. Resistor 22 can be used for added hysteresis. The gains are set that when spot 10 is white or yellow, the output of amplifier 18 will be high, as both detectors receive about the same amount of white light from fiber 14 but detector 16 only sees part of this light (the red component). When spot 10 is red, all the returned light is red and detector 16 sees about as much light as detector 15, causing a larger signal at the inverting input of amplifier 18, pulling the output to zero. This allows the user to determine if blood is present and enable the “OK” LED only when both parameters (color and electrical properties) are the desired values. In case of blood detecting version, LED will turn on only when the electrical resistance is low and blood was not detected. In case of pH detecting version, LED will turn on when electrical resistance is low and a red color was detected. The disclosed invention can use any combination, either whole or in part, of the methods described above to detect gastric acid and the absence of blood.

Clearly the methods of detecting blood and gastric acid can be miniaturized and placed inside the distal tip of NG tube 6.

Other electrical methods of detecting gastric acid can be used as well. By the way of example, a battery or voltaic cell can be formed from several layers of dissimilar metals separated by a porous material.

When gastric acid wets the battery, a voltage sufficient to light up an LED is created. A typical battery can be made of four cells in series, each cell having a thin foil of copper, about 4×20 mm, separated from a thin foil of zinc by tissue paper. The art of wet-cell construction is well known. The main problem of such batteries is the rapid decay of the output voltage, which can be overcome by introducing special chemicals, such as MnO2 and NH4Cl between the metals foils. Such chemicals absorb the gasses created by the electrochemical and chemical reactions.

A single wire system can also be used, using the patient's body as a return wire.

While the preferred embodiment of the invented provides visual feedback with an LED, the feedback can come in many different forms, such as an audio feedback.

An advantage to the disclosed inventions over previous methods is that it is inexpensive to make and use, and therefore can be treated as a disposable device, either manufactured as part of the NG tube or as a separate, insertable device. It can also be manufactured in such a way that the detection device can be used with disposable NG tubes.

Note that we use the terms NG tube, feeding tube and medical catheter interchangeably in this document.

1. A device for Nasogastric tube placement verification, wherein said verification is based on the presence of gastric acid and the absence of blood.

2. A device as in 1, wherein said gastric acid is detected with a continuity conductor.

3. A device as in 1, wherein a presence of blood is determined using the color of blood.

4. A device as in 1, wherein a presence of blood is based on the red colour of blood blocking blue light.

5. A device as in 1, wherein the gastric acid is detected with a voltaic cell.

6. A device as in 1, wherein a continuity circuit is completed when exposed to acid between a pH of 0 and 5.

7. A device as in 1, wherein said device can be removed from a Nasogastric tube.

8. A device as in 1, wherein a permission to feed verification is presented as either an audio or visual feedback, or a combination of both.

9. A device as in 1, wherein said device can be inserted into a medical catheter.

10. A device as in 1, wherein said device can be fully or partially affixed to a medical catheter.

11. A method for Nasogastric tube placement verification, wherein said verification is based on the presence of gastric acid and the absence of blood.

12. A method as in 11, wherein said gastric acid is detected using a continuity conductor.

13. A method as in 11, wherein a presence of blood is determined using the color of blood.

14. A method as in 11, wherein a presence of blood is based on the red colour of blood blocking blue light.

15. A method as in 11, wherein the gastric acid is detected with a voltaic cell.

16. A method as in 11, wherein a continuity circuit is completed when exposed to acid between a pH of 0 and 5.

17. A method as in 11, wherein said verification method can be removed from a Nasogastric tube.

18. A method as in 11, wherein a permission to feed verification is presented as either an audio or visual feedback, or a combination of both.

19. A method as in 11, wherein said verification method can be inserted into a medical catheter.

20. A method as in 11, wherein said verification method can be fully or partially affixed to a medical catheter.
